(19) United States
(12) Patent Application Publication
(10) Pub. No.: US 2009/0205668 A1
(43) Pub. Date: Aug. 20, 2009

(54) Condom with anti slippage feature

(76) Inventor: JEAN MORISSETTE, Chicoutimi (CA)

Correspondence Address: Inventarium, Suite 1607, 4050 Rosemont blvd, Montreal, QC, H1X 1M4, CA

(21) Appl. No.: 12/342,053

(22) Filed: Dec. 22, 2008

Related U.S. Application Data
(60) Provisional application No. 61/029,387, filed on Feb. 18, 2008.

(51) Int. Cl. A61F 6/04; A61B 19/00

(52) U.S. Cl. 128/844; 128/898

(57) ABSTRACT

An anti slippage condom has body and a scrotum retainer part. The scrotum retainer part extends integrally from the body and a slit delineates the body from the scrotum retainer part.

This application claims priority based on provisional 61029387 filed Feb. 18, 2008

### FIELD OF THE INVENTION

The present invention relates generally to prophylactic devices but more particularly to a condom with means of attachment.

### BACKGROUND OF THE INVENTION

Condoms have been in use for centuries and for that long, there has been the problem of the condom slipping out and sometimes staying in the vagina.

The prior art shows numerous patents as well as design patents that show condoms with strings or straps and related attachment means.

For example, U.S. Pat. No. 5,531,230 shows a condom that includes straps that extend from the proximal end of the condom and can be positioned about a waist of a male wearer to secure the condom from unintentional removal.

U.S. Pat. No. 5,666,971 shows a device for improving erection, having a ring-shaped base having two walls spaced from one another to define a cavity therebetween, a cylindrical elastic closed cover connected to the base and having a portion which is arcuate-shaped and adapted to cover the head of the penis. A longitudinal portion which is adapted to cover the shaft of the penis.

U.S. Pat. No. 6,308,709 also shows an erection-facilitating condom for treatment of erection dysfunction. The erection-facilitating condom includes a flexible tubular membrane having a closed end and an open end; and also includes a ring member being attached to the flexible tubular membrane; and further includes a flexible line being connected to the ring member for closing the flexible tubular member tightly about a sex organ of a user.

The drawback of the prior art is that it is mostly concerned with maintaining an erection and as such, would not be efficient at preventing slippage. In the case of U.S. Pat. No. 5,666,971, the straps need to be very long in order to wrap around the waist.

There is thus a needed for an economically produced and efficient anti slippage condom.

### SUMMARY OF THE INVENTION

In view of the foregoing disadvantages inherent in the known devices now present in the prior art, the present invention, which will be described subsequently in greater detail, is to provide objects and advantages which are to provide for a means for keeping the condom on the penis even after removal from the vagina.

It is another advantage of this invention to squeeze the area behind the testicles to provide a better, more sustained erection

To attain these ends, the present invention generally comprises a body and a scrotum retainer part. The scrotum retainer part extends integrally from the body and a slit delineates the body from the scrotum retainer part.

Preferably, the slit has non-tearing means.

In a preferred embodiment, a base ring is located at the base of the scrotum retaining part and the base ring provides comparatively reduced elasticity in relation to the scrotum scrotum retainer part and the body so as to retain the scrotum retaining part behind the scrotum.

The slit comprising more than half of the circumference of the condom.

The slit non tearing means consists of having the ends of the slit terminated by holes.

Alternatively, the slit non tearing means can consist in having the ends of the slit terminated by increased thickness.

In yet another variant, the slit non tearing means can have the ends of the slit its terminated by increased thickness and holes.

The anti slippage condom has a method for putting it on which consists in in the steps of a user unrolling the condom over its penis by making note of the location of the slit; positioning the slit so as the locate the slit under the penis; and pulling the base ring so that it pulls the scrotum retained part behind the scrotum.

There has thus been outlined, rather broadly, the more important features of the invention in order that the detailed description thereof that follows may be better understood, and in order that the present contribution to the art may be better appreciated. The term condom, improved condom, anti-slipping, and anti slippage condom are used throughout to describe the invention and it should be understood that they are to be used interchangeably to define the same invention. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S.

Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be made to the accompanying drawings and descriptive matter which contains illustrated preferred embodiments of the invention.

### BRIEF DESCRIPTION OF THE DRAWINGS

FIGS. 1a-b Perspective views with the condom in various stages of installation.

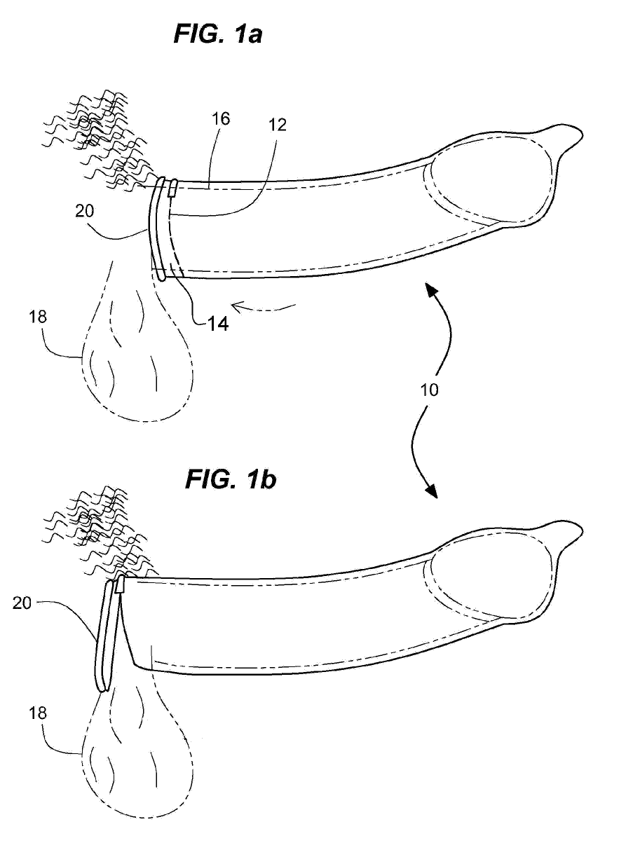

FIGS. 2a-d Side elevation and side elevation detail, as well as flattened views of the slit, respectively.

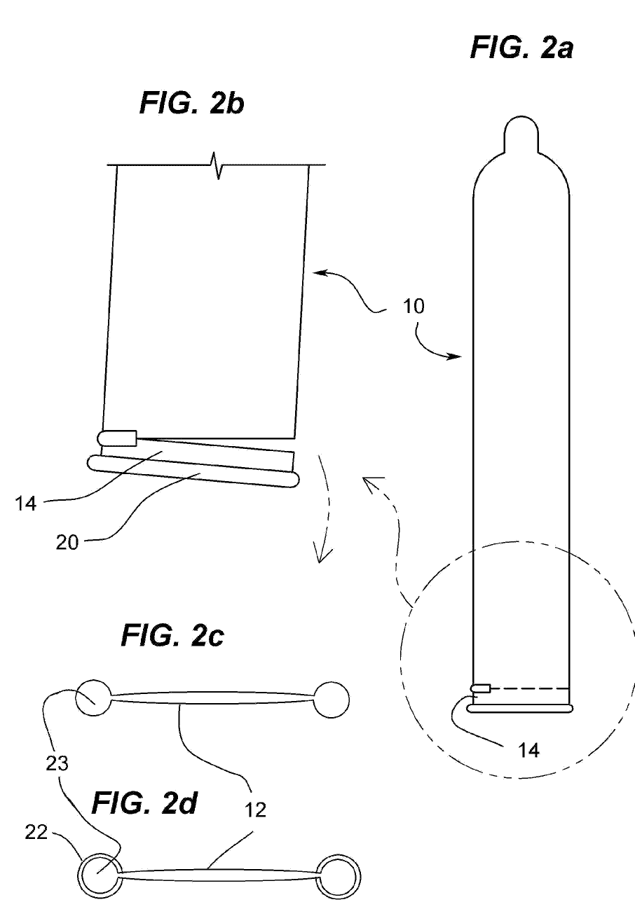

FIGS. 3a-b Side elevation and cross section along B-B of 3a, respectively.

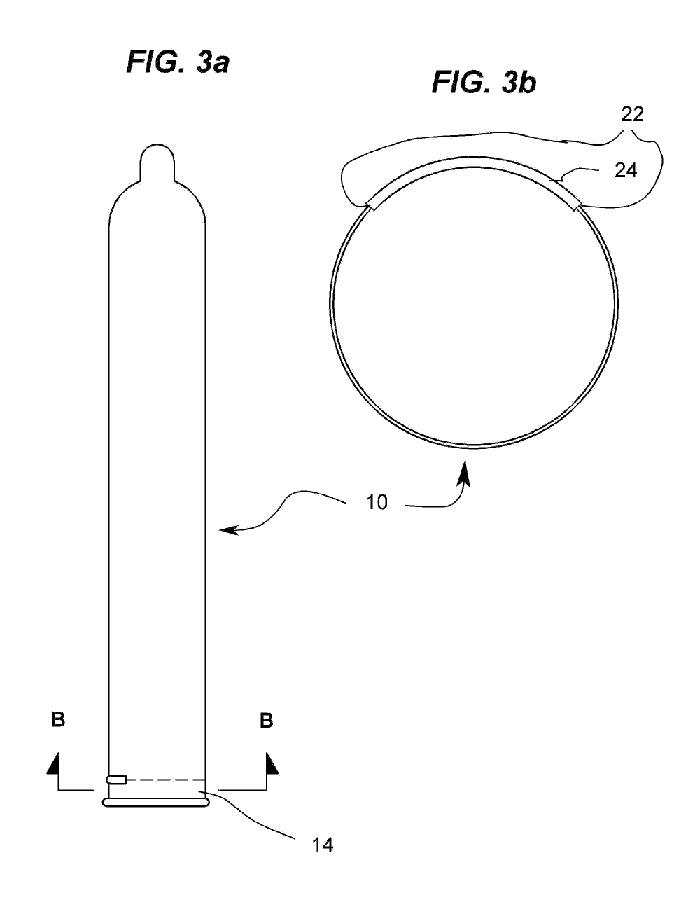

### DETAILED DESCRIPTION

An improved condom (10) comprises the general features commonly found on existing condoms as far as shape and material are concerned but comprises a slit (12) which partially opens an area defined as “scrotum retained part” (14) and which is located proximal the base (16) of the condom (10). The slit (12) allows for the scrotum retained part (14) to be placed behind the scrotum (18) so that the scrotum (18) itself will hold, or retain, the scrotum retained part (14). A base ring (20) acts like a tight elastic which further helps the scrotum (18) to retain the scrotum retained part (14) and thus, the condom (10).

The slit (12) generally comprises over half of the circumference of the condom (10) and has its ends (22) created, during the manufacturing process, in such a way so as to prevent any expansion of the slit (12) through involuntary tearing. Given the fragile nature of latex condoms, this is important. Various means can be employed such as terminating the ends (22) with holes (23) which diffuse pressure conducive to tearing, or by making the latex of an increased thickness (24) in the manufacturing process. Or, a combination of both the holes (23) and increased thickness (24) around the periphery of the holes (23).

There could be other means such as special treatment of the latex to alter its properties to disallow further tearing.

In order to use the condom (10), a user would unroll it over its penis, as is known in the art, but making note of the location of the slit (12) so that it is located under the penis. Finish by pulling the base ring (20) so that it pulls the scrotum retained part (14) behind the scrotum (18). To help in locating the position of the slit (12), an indicia (not shown) is placed on the condom (10) so as to visibly indicate the position of the slit (12) before the condom is unrolled.

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. An anti slippage condom comprising:
a body and a scrotum retained part;
the scrotum retained part extending integrally from the body;
a slit delineating the body from the scrotum retained part.

2. The anti slippage condom of claim 1 wherein:
the slit has non-tearing means.

2. The anti slippage condom of claim 1 wherein:
a base ring located at the base of the scrotum retained part;
the base ring providing comparatively reduced elasticity in relation to the scrotum scrotum retained part and the body so as to retain the scrotum retained part behind the scrotum.

3. The anti slippage condom of claim 1 wherein:
the slit comprising more than half of the circumference of the condom.

4. The anti slippage condom of claim 2 wherein:
the slit non tearing means consisting of having the ends of the slit terminated by holes.

5. The anti slippage condom of claim 2 wherein:
the slit non tearing means consisting of having the ends of the slit terminated by increased thickness.

6. The anti slippage condom of claim 2 wherein:
the slit non tearing means consisting of having the ends of the slit terminated by increased thickness and holes.

7. A method of putting on the anti slipping condom of claim 1 consisting in the steps of:
a user unrolls the condom over its penis by making note of the location of the slit;
positioning the slit so as the locate the slit under the penis;
pulling the base ring so that it pulls the scrotum retained part behind the scrotum.
